(19) United States
(12) Patent Application Publication
(10) Pub. No.: US 2011/0172394 A1
(43) Pub. Date: Jul. 14, 2011

(54) IMPLANTABLE MATERIAL FOR THE REPAIR, AUGMENTATION, OR REPLACEMENT OF BONE AND A METHOD FOR THE PREPARATION THEREOF

(75) Inventors: David Knight, Hampshire (GB); Nicholas Skaer, Oxfordshire (GB); Andrew Collins, Somerset (GB); Tom Louis Dirk Gheysens, Oxfordshire (GB)

(73) Assignee: ORTHOX LIMITED, Abingdon, Oxfordshire (GB)

(21) Appl. No.: 13/001,287

(22) PCT Filed: Jun. 24, 2009
(86) PCT No.: PCT/GB2009/050727

§ 371 Date: Mar. 24, 2011

(30) Foreign Application Priority Data
Jun. 24, 2008 (GB) 0811542.0

(51) Int. Cl. C07K 14/435

(52) U.S. Cl. 530/353

(57) ABSTRACT

A method for the preparation of an implantable material for the repair, augmentation or replacement of bone from a fibroin solution, the method comprising the steps of: preparing a gel from the fibroin solution; preparing a material by subjecting the gel to one or more steps of freezing and thawing the gel, wherein the step of preparing the gel from the fibroin solution is performed in the presence of phosphate ions. The material may be treated with calcium ionstoform a fibroin-apatite. A further method comprises the step of treating the material with an isocyanate. The invention also extends to a method for the preparation of an implantable material, wherein a regenerated fibroin solution is used. Also, there is an implantable material and an implant.

### TECHNICAL FIELD

The present invention relates generally to an implantable material and a method for the preparation thereof. The material is useful, for example, for the repair, augmentation, or replacement of substantially all or part of one or more bones, or as a substitute for bone grafts in orthopaedic applications.

### BACKGROUND OF THE INVENTION

Except where specified below the term ‘fibroin’ is used to refer generically to the main structural protein of cocoon silks whether they are derived from the domesticated Mulberry Silkworm (Bombyx mori), a transgenic silkworm or from any Wild Silkworm including, but not limited to those producing Muga, Eri or Tussah silks.

Furthermore, the term ‘silk’ is used to refer to the natural fine fibre that silkworms secrete, which mainly comprises the two proteins, fibroin and sericin, fibroin being the principal structural material in the silk, and sericin being the material surrounding the fibroin and sticking the fibres together in the cocoon.

‘Silk cocoon’ is used to refer to the casing of silk spun by the larvae of the silk worm for protection during the pupal stage.

The term ‘bone repair’ refers to any procedure for repairing bone, including those which use a material as a substitute for bone grafts.

The term ‘bone augmentation’ refers to the use of any procedure for adding or building bone.

The term ‘bone replacement’ refers to the use of any procedure for replacing existing bone.

The term ‘polymer’ is used to refer to all large molecules comprised of chains of one or more types of monomeric units and includes macromolecular proteins.

There are a number of injuries and conditions that require surgical intervention to repair, augment, or replace substantially all or part of one or more bones. These conditions include, for example, traumatic fractures, non-unions, bone cysts, critical bone defects, loosening of prostheses at the bone/prosthesis interface and malignant tumours in bone.

Historically, many of these conditions could only be repaired by autografts (where tissue is transplanted from one part of the body to another in the same individual, also called an autotransplant), or allografts (where an organ or tissue is transplanted from one individual to another of the same species with a different genotype, also called an allogeneic graft or a homograft) using materials derived from bone.

Autografts are currently the favoured option for bone repair. However autografting has several associated problems, including the high costs for the surgical harvesting procedure and pain and morbidity experienced at the harvest site. For example, harvesting a graft from the iliac crest, the protruding bony section of the patient's hip, can cost between $1,000 to $9,000 per procedure for the harvesting operation and the additional hospital stay. Where morbidity is experienced at the harvest site, symptoms include pain, infection, nerve damage and blood loss, the latter often requiring blood transfusion associated with the risk of blood borne infection. The quantity of bone tissue that can be harvested is limited and can be of poor quality especially in osteoporotic patients.

Allograft materials taken from cadavers circumvent some of the shortcomings of autografts by eliminating donor site morbidity and issues of limited supply as taught by Burkuss, J. K. (2002) in his article “New Bone Graft Techniques and Applications in the Spine” in Medscape today (http://www.medscape.com/viewarticle/443902). However, the use of allografts presents additional risks and problems not seen with autografts. In an allograft, because the tissue is obtained from a donor, there is a risk of disease transmission from donor to recipient and it has been established that HIV/hepatitis can be transmitted through allografts. In addition, allografts and allogenic implants are acellular and are less successful and less predictable than autografts for reasons attributed to immunogenicity and the absence of viable cells that become osteoblasts.

Due to the shortcomings of autografts and allografts, efforts have been made to find suitable bone repair materials (BRMs) for use as alternatives to autografts and allografts. However, BRMs have not yet replaced autografts, because in the past they have failed to adequately address five main criteria: load bearing ability; osteoconductivity; osteoinductivity; resorbability (as taught by Rose, F. R. A. J., and Oreffo, R. O. C. (2002) in their article “Bone Tissue Engineering: Hope vs Hype.” Published in Biochem. Biophys. Res. Commun 292, 1-7); and ease of use in theatre. Ease of use in theatre is of considerable importance and is not met by many artificial BRMs.

Ideally, BRMs need to be able to be capable of full and immediate load-bearing. In this context, load-bearing can be defined as the ability of a BRM to maintain its mechanical integrity without undue distortion when subjected to the forces applied to it in the course of normal everyday life without recourse to secondary supporting structures, such as pins, plates, external fixators, and casts. Furthermore, immediate load bearing can be defined as the ability of the repair to bear full loads by the time the patient has recovered from anaesthesia.

The material properties that enable immediate load bearing of the BRM depends on the location into which the BRM is to be implanted, but includes good compressive toughness, good compressive strength, good compressive elastic modulus and good interfacial properties with the existing bone. It is clear that the minimum requirement for immediate load bearing is for the strength and toughness of the material to match that of healthy bone at the site of implantation. Furthermore, it is generally understood that BRMs need to mimic the properties of bone fairly closely to prevent high local stress concentrations or stress shielding, both of which are likely to adversely affect natural bone adjacent to the implanted BRM. Thus it is highly desirable to use the mechanical properties of normal bone as target values for load bearing BRMs.

Toughness provides resistance to fracture and is extremely important in bone. Toughness is measured in units of joules per cubic metre (Jm⁻³). There are several methods for measuring the toughness of bones and the values obtained depend to an extent on the method that is used and the exact conditions of specimen loading. However, for a mid-diaphyseal femur of a healthy 35 year old, the work of fracture method, the impact of notched bone method and the J-integral method all gave similar results of 3.9 kJ m⁻³, 2.0 kJ m⁻³ and 1.3 kJ m⁻³, respectively (disclosed by Zioupos, J. in his article, “Ageing human bone: factors affecting its biomechanical properties and the role of collagen” published in the Journal of Biomaterials (applied) (2001) 15, 187-231). Furthemore, a value of about 1 kJ m⁻³ for the toughness of bone was provided in studies conducted by Ashby, M F; Gibson, L J; Wegst, U; and Olve, R. in their metanalysis published in Proceedings of the Royal Society, Mathematical and Physical Sciences (1995), 450, 123-140. Thus a target compressive toughness of 1.3 kJ m⁻³ measured by the J-integral method is appropriate for load-bearing BRMs.

The compressive strength of normal human cancellous bone shows considerable variation, but typically is about 5 MPa, though may fall beneath 2 MPa in osteoporotic bone (Togawa, D. Kayanja, M. M., and Lieberman, I. H. (2005), “Percutaneous Vertebral Augmentation” in The Internet Journal of Spine Surgery 1, (2), http://www.ispub.com/ostia/index.php?xmlFilePath=journals/ijss/vol1n2/vertebral.xml).

Cortical bone, with a compressive strength of about 10-160 MPa, is considerably stronger than cancellous bone (Cowin, S. Ed (1989) “Bone Biomechanics”. CRC Press, Boca Raton and by Duck, F. A. (1990) “Physical Properties of Tissue: A comprehensive Reference Book”, Academic Press, London). Although cortical bone is often much thinner than the underlying trabecular bone, it makes a significant contribution to the mechanical properties of whole bone, accounting for approximately 60% of the bending strength in the femoral neck and about 10% of the compressive strength of vertebral bodies (Werner et al., 1988). Thus a target compressive strength of about 20 MPa is appropriate for load bearing BRMs.

An approximate match between the compressive elastic modulus of a BRM and bone is also important to prevent high stress accumulation and stress shielding. Cortical bone has an elastic modulus of 12-18 GPa while that for cancellous bone is 0.1-0.5 GPa (Rezwana, K.; Chena, Q. Z.; Blakera, J. J.; Boccaccini, A. R., (2006), “Biodegradable and bioactive porous polymer/inorganic composite scaffolds for bone tissue engineering.” in Biomaterials, 27 3413-3431). As most of an implant of a BRM will be in contact with cancellous bone rather than cortical bone, a compressive elastic modulus of 0.1-0.5 GPa is an appropriate target for BRMs.

Solid hydroxyapatite, bioglass or glass-ceramic mixtures are considerably stiffer than bone, while porous hydroxyapatite is considerably less stiff, as disclosed by Rezwana, K (2006) op. cit.

It is generally understood that mineral density is a major determinant of compressive strength and compressive elastic modulus in mineralized composites. Thus, the compressive strength and compressive elastic modulus of trabecular bone increases approximately with the square of its density (Carter, D. R. and Hayes, W. C., (1976) in the article “Bone compressive strength: the influence of density and strain rate” published in Science 194, 1174-1176). This may also be true for ceramic and for mineral-containing composite BRMs. Thus, it is highly desirable from a mechanical perspective that composite BRMs are heavily mineralised.

In addition to the requirement that the mechanical properties should match those of the bone, BRMs need to be osteoconductive. Osteoconductivity is generally defined as the process by which osteogenic cells migrate to the surfaces of a material through the fibrin clot established immediately after implantation of a BRM. This migration of osteogenic cells through the clot causes retraction of the temporary fibrin matrix. Hence, it is important that the fibrin matrix is well secured to the material, because if it is not, when osteogenic cells start to migrate along the fibrin fibres, wound contraction can detach the fibrin from the material. It has been previously shown that a rough surface will bind the fibrin matrix better than a smooth surface and hence will facilitate the migration of osteogenic cells to the surface of the material.

Therefore, it is generally accepted that the factors that are important for osteoconductivity are as follows:

(i) an open porous structure with pores of sufficient size to allow the migration of bone-forming cells, whilst preventing the migration of other tissues and unwanted cell types;

(ii) provision of some pores of sufficient size to allow for the inward migration of blood vessels;

(iii) maintenance of a suitable vascularised environment for bone cell differentiation;

(iv) provision of a suitable surface for bone cells adhesion and function; and

(v) a rough surface to bind the fibrin matrix.

Thus, a porous structure is highly desirable to enable cells and new vessels to colonise the interior of the porous BRM. The minimum pore size to permit cellular ingress is considered to be 100 μm, but pore sizes of 300 μm may enhance vascularisation and new bone formation and smaller pores favor hypoxic conditions and cartilage formation before osteogenesis (Karageorgiou, V.; Kaplan, D. (2005), “Porosity of 3D biomaterial scaffolds and osteogenesis” in Biomaterials, 26, (27), 4745491). However, greater pore size and porosity have a negative effect on the compressive strength, compressive elastic modulus and compressive toughness of a BRM.

A range of methods have been used to produce intercommunicating pores in materials including thermally induced phase separation, freezing, solvent casting, particle leaching, supercritical gas foaming, incorporation of resorbable monofilaments, sintering of microsphere and solid free form coating. Many proposed BRMs either lack pores completely or have pores of an inappropriate size for optimal osteoconductivity.

Osteoinductivity is generally defined as the ability to induce non-differentiated stem cells or osteoprogenitor cells to differentiate into osteoblasts. The simplest test of osteoinductivity is the ability to induce the formation of bone in tissue locations such as muscle which do not normally form bone (ectopic bone growth). Some allograft substitutes are osteoinductive, probably on account of the bound growth factors. Some calcium phosphate minerals are osteoinductive possibly because they adsorb and concentrate bone growth factors from tissue fluids. It is generally understood that a variety of BRMs can be made osteoinductive by adding growth factors such as rhBMP-2 to them.

It is generally understood that it is highly desirable that BRMs are fully resorbable to allow entire BRM replacement with endogenous tissue. It is also generally understood that in a load bearing BRM, the half-resorption time needs to be fairly slow, probably about 9 months, to allow time for the replacement tissue to acquire full strength and toughness to take over load-bearing from the BRM. Synthetic polymers based on monomers of lactic acid, glycolic acid, dioxanone, trimethylene carbonate and caprolactone, or a combination of these monomers resorb too quickly and have acidic breakdown products which may be irritants.

Currently there are no existing products on the market that fulfill the main criteria for the ideal BRM as stated by Rose and Oreffo (2002), op. cit. Existing load-bearing BRMs comprising mineral and resin composite, bioglass, or metal are not absorbed and remain in situ at the graft site in perpetuity. It is generally accepted that this can result in a modulus mismatch leading to high stress concentrations and stress shedding leading to bone resorption. This can cause loosening of the implant and consequently contribute to the failure of the implant to fully integrate with the endogenous tissue. Non-resorbable implant materials may also serve as foci for infection and irritation. Eventual mechanical failure of non-resorbable bone implants may require them to be replaced by surgery leading to concomitant pain, risk of infection and further expense.

Materials containing calcium phosphate are still a long way from reaching the acceptable allograft standard as stated by Tas, A. C., in “Participation of calcium phosphate bone substitutes in the bone remodeling process: Influence of materials chemistry and porosity”, published in Euro Ceramics Viii, Pts 1-3, 2004; Vol. 264-268, pp 1969-1972.

Non-load bearing BRMs require secondary support mechanisms over the entirety of the healing period, in some cases for periods in excess of six months, to allow successful union of the fractured surfaces across the graft. Non-load-bearing materials are only used in a comparatively small number of instances in which load-bearing is not required.

WO 2005/094911A2 discloses a composite material comprising one or more silk elements in an acrylic or cross-linked protein matrix. The silk elements are made from the group of silk, elements consisting of domestic silkworm silk, wild silkworm silk, spider dragline silk, and filaments spun from recombinant silk protein or protein analogues. The composite material is particularly useful for use in surgical implants. The fibroin matrix disclosed was prepared from regenerated silk fibroin made according to what is widely accepted as the ‘standard protocol’ for preparing regenerated silk fibroin, as disclosed in literature (Chen, X., Knight, D. P., Shao, Z. Z., and Vollrath, F. (2001) “Regenerated Bombyx silk solutions studied with rheometry and FTIR” Polymer, 42, 9969-9974). The standard protocol for preparing regenerated fibroin solutions involves degumming in hot (typically 100° C.) alkaline solutions and dissolution in hot 9M to 9.5M lithium bromide solution for periods of time in excess of 24 hours. It has been found that the standard protocol for preparing regenerated silk fibroin does not result in sufficient strength, toughness and stiffness to confer immediate and full load bearing.

WO 2007/020449 A2 discloses a cartilaginous tissue repair device with a biocompatible, bioresorbable three-dimensional silk or other fibre lay and a biocompatible, bioresorbable substantially porous silkbased or other hydrogel, partially or substantially filling the interstices of the fibre lay, with or without an integral means of firmly anchoring the device to a patient's bone. The application discloses the use of acylating agents including aliphatic and bifunctional isocyanates, dodecyl isocyanate or hexamethylene diisocyanate to increase the hydrophobicity of the material.

PCT/IB2009/051775 discloses an implantable material and a method for the preparation thereof wherein the material is prepared from an optimized regenerated fibroin solution. The implantable material can be used for the replacement, partial replacement, repair or augmentation of human cartilage. The implantable material has an unconfined compressive tangent modulus at 10% strain of between 0.3-5.0 MPa, an ultimate compressive strength (stress to yield point) of up to 20 MPa, is substantially resilient, has an open porosity with pore size ranging from 20 to 1000 μm and is slowly resorbable.

The use of solutions of aromatic isocyanates in dry pyridine to cross-link proteins including silk fibroin threads was first disclosed by Fraenkel-Conrat, H.; Cooper, M.; Olcott, H. S. 1945, “Action of Aromatic Isocyanates on proteins”, Journal of the American Chemistry Society, 67, 314. This disclosure built on the work of Farnworth, A. (1955), “The Reaction Between Wool and Phenyl isoCyanate” Biochemistry Journal, 59, 529, which disclosed the use of phenyl isocyanate in dry pyridine to cross-link wool extensively.

More recently, the effect of cross-linking of natural silk fibroin threads by different isocyanates has been investigated by Arai, T, Ishikawa, H., Freddi, Winkler, G S and Tsukada, M (2001), “Chemical modification of Bombyx mori silk using isocyanates”, Journal of Applied Polymer Science, 79, 1756-1763. The fibres were first swollen in dimethylsulphoxide or dimethylformamide and then treated with an isocyanate in the same solvent. Different isocyanates produced different increases in fibre mass and the tensile strength declined slightly in proportion to the mass gain of the fibre. Threads treated with phenyl isocyanate in dimethylsulphoxide for different periods of time actually produced a marked and progressive decrease in tensile strength and elongation to break. Thus, a person skilled in the art would not expect that a reagent that actually reduced the tensile strength and stiffness of silk fibres might be useful for increasing the compressive strength and stiffness of porous materials prepared from regenerated silk fibroin.

U.S. Pat. No. 6,902,932 discloses a silk-fiber-based matrix having a wire-rope geometry for use in producing a ligament or tendon, particularly an anterior cruciate ligament, ex vivo for implantation into a recipient in need thereof. The document further discloses a silk-fiber-based matrix which is seeded with pluripotent cells that proliferate and differentiate on the matrix to form a ligament or tendon ex vivo. Also disclosed is a bio-engineered ligament, comprising a silk-fiber-based matrix seeded with pluripotent cells that proliferate and differentiate on the matrix to form the ligament or tendon. Finally, a method for producing a ligament or tendon ex vivo comprising a silk-fiber-based matrix is also disclosed. The material is designed for use as a scaffold for cells and would not be load-bearing when used for bone repair.

US 2006/0095137 discloses the use of non-woven silk fibroin fibers which can contain a ceramic. The material can be used for guided bone tissue regeneration. The material is only intended to guide bone tissue regeneration and is not for use as a load-bearing BRM. The material is highly unlikely to be load-bearing at the time of implantation and no evidence for load-bearing capability is presented.

US 2007/0187862 discloses the use of a fibroin solution concentrated by reverse dialysis against a hygroscopic polymer and the production of a foam using salt particles and/or by bubbling gas through the solution.

US 2007/0187862, WO2005/012606, EP1613796 and CA2562415 disclose a porous fibroin scaffold that can contain appropriate signal factors including bone morphogenic protein, which can be seeded with bone stromal cells. In one aspect of the invention, the three-dimensional porous silk scaffold can itself be implanted in vivo and serve as tissue substitute for bone. However, the material cannot be considered to be load-bearing at the time of implantation and no evidence for load-bearing capability is presented.

It is therefore, an object of the present invention to provide an implantable bone repair, augmentation, or replacement material and a method of preparing the material, where the material has improved mechanical properties.

It is a further object of the invention to provide an implant for the total or partial replacement, augmentation or repair of bone.

### SUMMARY OF THE INVENTION

According to a first aspect of the present invention there is provided a method for the preparation of an implantable material for the repair, augmentation or replacement of bone from a fibroin solution, the method comprising the steps of:
preparing a gel from the fibroin solution; and
preparing a material by subjecting the gel to one or more steps of freezing and thawing the gel,

wherein the step of preparing the gel from the fibroin solution is performed in the presence of phosphate ions.

The fibroin solution may be dispersed with phosphate ions before the step of preparing the gel from the fibroin solution. The step of preparing the gel from the fibroin solution may comprise treating the fibroin solution with an alkaline solution. Preferably, the dispersal of the phosphate ions in the fibroin solution comprises phosphate ions in an aqueous buffer at a neutral pH.

Most preferably, the step of preparing the gel from the fibroin solution comprises a gelling reagent containing phosphate ions. Particularly good results have been observed when the fibroin solution is gelled using an aqueous buffered solution of dihydrogen sodium phosphate adjusted to an alkaline pH.

The step of preparing the gel from the fibroin solution may comprise, for example, subjecting the solution to microwave radiation, sound, infra-sound or ultrasound, laser radiation mechanical shearing or rapid extensional flow or acidic solutions or vapours.

The step of preparing the gel from the fibroin solution may be performed at any suitable temperature, for example, within a temperature range of approximately 0° C. to approximately 30° C. for a period of, for example, approximately 2 hours, where the step of preparing the gel from the fibroin solution is performed on 20 ml of fibroin solution in a Visking bag with a 0.9 M solution of dihydrogen sodium phosphate surrounding the bag.

The gelling time may be determined based upon the depth of penetration of the gellation required.

The methods may comprise inserting one end of a bone anchoring device into the fibroin solution prior to the step of preparing the gel from the fibroin solution. The bone anchoring device may comprise a plurality of braided or twisted fibres or threads, or a cable.

Freezing of the gel may be performed at any suitable temperature, for example, within a temperature range of approximately −1° C. to approximately −120° C. Preferably, freezing is performed within a temperature range of approximately −10° C. to approximately −30° C. For example, good results have been achieved where freezing is performed at a temperature of approximately −13° C.

A plurality of freezing and thawing cycles may be performed to increase the diameters of the pores. Good pore sizes have been observed with up to five freeze/thaw cycles at −13° C.

The material may be treated with calcium ions to form a fibroin-apatite before treating the material with the isocyanate. The formation of a fibroin-apatite may comprise treatment of the material with either one of, or a mixture of calcium chloride and calcium nitrate to form a fibroin-chlorapatite, or a fibroin-hydroxyapatite, or a mixture of fibroin-chlorapatite and fibroin-hydroxyapatite.

Preferably, the calcium ions may be provided by an aqueous solution of calcium chloride. Other suitable aqueous solutions may comprise, for example, calcium nitrate.

Preferably, the material is treated with calcium ions at a basic pH. Most preferably, the material is treated with calcium ions at a pH of between approximately 7.0 and approximately 10.0. Good results have been achieved when the material is treated with calcium ions at a pH of approximately 9.0.

Excess calcium chloride, or other suitable calcium ion containing salt, may be removed from the material. The material may also be treated to convert the fibroin into the silk II state. For example, the material may be washed with ethanol to remove excess calcium chloride and to convert the fibroin into a silk II state.

The material may be dried after the washing step. Drying may be by heat drying, air drying, or any other suitable method. Good results have been observed using vacuum drying.

The material may be treated with a cross-linking agent. By treating the material with a cross-linking agent, cross-links are formed between the fibroin polymers in the material. The cross-links between the fibroin polymers may be covalent cross-links.

The material may be treated with any suitable cross-linking agent. Suitable cross-linking agents may include, for example, an isocyanate, a carbodiimide, or a cyanoacrylate.

Suitable carbodiimides may include EDC (1-ethyl-3-(3-dimethylaminopropyl)carbodiimide), or N,N′-dicyclohexylcarbodiimide Suitable cyanoacrylates may include methyl 2-cyanoacrylate, ethyl-2-cyanoacrylate, n-butyl cyanoacrylate and 2-octyl cyanoacrylate. Where a cyanoacrylate is used, cross-linking may be performed under inert atmospheric conditions to prevent solidification.

Preferably, the cross-linking agent is an isocyanate. Preferably, the isocyanate is a di-isocyanate. Suitable di-isocyanates may include one or more of hexamethylene di-isocyanate (HDI), methylene diphenyl di-isocyanate (MDI), toluene di-isocyanate (TDI) and isophorone di-isocyanate (IPDI). Good results, for example, have been obtained using hexamethylene di-isocyanate.

Alternatively, the isocyanate may comprise a mono-isocyanate with an additional functional group. A suitable additional functional group may, for example, comprise a gluteraldehyde group.

Particularly good results have been obtained where the treatment with the cross-linking agent is carried out with substantially no fibroin swelling agents, such as water, dimethylsulphoxide or dimethylformamide. Preferably, the treatment with the cross-linking agent is carried out with no fibroin swelling agents.

By treating the material with the cross-linking agent in the absence of fibroin swelling agents, or with substantially no fibroin swelling agents, the separation of calcium and phosphate from the fibroin is reduced, or prevented.

Good results have been achieved where the material is treated with undiluted dry hexamethylene di-isocyanate at approximately 80° C. using dry nitrogen.

The method may comprise the step of varying the length of exposure of the material to the cross-linking agent to tune the density of the cross-linking and therefore, achieve the required stiffness and resorbability of the implantable material.

The method may comprise the further step of removing excess cross-linking agent from the material. The methods may therefore, comprise one or more rinsing steps using, for example, anhydrous acetone.

The method may also comprise one or more steps to hydrolyse excess CNO groups. This may be achieved by rinsing the material in water.

The method may further comprise the step of drying the material, by any suitable drying method, although preferably by heat drying.

The material may be sterilised by any suitable method, including, for example, autoclaving, exposure to gamma radiation or treatment with ethylene dioxide.

The fibroin solution may be a regenerated fibroin solution.

The regenerated fibroin solution may be prepared by a method comprising treating silk or silk cocoons with an ionic reagent comprising an aqueous solution of monovalent cations and monovalent anions, the cations and anions having ionic radii of at least 1.05 Angstroms and a Jones-Dole B coefficient of between −0.001 and −0.05 at 25° C.

As will be readily understood by those skilled in the art, the B coefficient of the Jones-Dole equation (Jones, G., and Dole, M., J. Am. Chem. Soc., 1929, 51, 2950) is related to the interaction between ions and water and is interpreted as a measure of the structure-forming and structure-breaking capacity of an electrolyte in solution.

Preferably, the cations and anions have a Jones-Dole B coefficient of between −0.001 and −0.046 at 25° C. More preferably, the cations and anions have a Jones-Dole B coefficient of between −0.001 and −0.007 at 25° C.

The method of preparing the regenerated fibroin solution may comprise degumming the treated silk or silk cocoons before, after or at the same time as the treatment of the silk or silk cocoons with the ionic reagent.

It is particularly preferred that the method comprises a further step of drying the silk or silk cocoons after treatment of the silk or silk cocoons with the ionic reagent. Preferably, the drying step is performed consecutively after the step of treatment with the ionic reagent. Most preferably, the drying step is performed after both the treatment with the ionic reagent and the degumming step has been performed.

The aim of the drying step is to extract as much water as possible from the treated silk or silk cocoons. Ideally, substantially all of the water is removed from the treated silk or silk cocoons.

The process of drying the silk or silk cocoons may be performed by, for example, air drying, freeze drying, or drying through the application of heat. Preferably, the step of drying the silk or silk cocoons comprises air drying.

The silk or silk cocoons may be dried at any suitable temperature. For instance, good results have been observed by drying the silk or silk cocoons at room temperature (21° C.).

The silk or silk cocoons may be dried over any suitable time period. Typically, the silk or silk cocoons may be dried for a period of several hours, for example 12-16 hours.

In some embodiments, the silk or silk cocoons may be air dried in conditions of less than 20% humidity. Preferably, drying of the silk or silk cocoons is carried out in the presence of a desiccant, which may include anhydrous calcium chloride or other suitable desiccants. Other suitable desiccants may include silica gel, calcium sulfate, calcium chloride and montmorillonite clay. Molecular sieves may also be used as desiccants.

The ionic reagent may comprise a hydroxide solution. The hydroxide solution may be formed in situ. For example, the silk or silk cocoons may be treated with ammonia gas or vapour to form ammonium hydroxide in combination with water already present in the silk or silk cocoons. Furthermore, water vapour may be added to the silk or silk cocoons either before the ammonia gas or vapour, with the ammonia gas or vapour, or subsequently.

Suitable ionic reagents include aqueous solutions of ammonium hydroxide, ammonium chloride, ammonium bromide, ammonium nitrate, potassium hydroxide, potassium chloride, potassium bromide, potassium nitrate, rubidium hydroxide, rubidium chloride, rubidium bromide and rubidium nitrate.

The ionic reagent functions to increase the solubility of proteins in the silk by increasing the charge density on the protein (‘salting in’).

The method may comprise a subsequent step (c) of dissolving the degummed silk or silk cocoons in a chaotropic agent.

The step of dissolving the silk or silk cocoons in the chaotropic agent may be performed under any one of the following conditions, or any combination of the following conditions:

at a temperature of less than 60° C.;

with a concentration of chaotropic agent up to 9.5M; and

for a period of time of less than 24 hours.

The degummed silk or silk cocoons may be dissolved in the chaotropic agent at any suitable temperature, for example, within a temperature range of approximately 10° C. to approximately 60° C. For instance, the degummed silk or silk cocoons are dissolved in the chaotropic agent within a temperature range of approximately 15° C. to approximately 40° C. Good results have been achieved by dissolving the degummed silk or silk cocoons in the chaotropic agent at a temperature of approximately 37° C.

The degummed silk or silk cocoons may be dissolved in the chaotropic agent at any suitable concentration, for example, in a concentration of the chaotropic agent of 9.3M. For instance, the degummed silk or silk cocoons may be dissolved in a concentration of the chaotropic agent of less than 9M. The degummed silk or silk cocoons may be dissolved in the chaotropic agent at a concentration of chaotropic agent within the range of approximately 6M to 9M, for example, approximately 7M.

The degummed silk or silk cocoons may be dissolved in the chaotropic agent for any suitable time period, for example, a time period of less than 24 hours. The degummed silk or silk cocoons may be dissolved in the chaotropic agent for a period of time of less than 12 hours. Preferably, the degummed silk or silk cocoons are dissolved in the chaotropic agent for a period of time of 4 to 5 hours and most preferably for less than 4 hours.

The chaotropic agent may comprise one suitable chaotropic agent or a combination of suitable chaotropic agents. Suitable chaotropic agents include lithium bromide, lithium thiocyanate, or guanidinium thiocyanate. A preferred the chaotropic agent comprises an aqueous lithium bromide solution.

Degumming the silk or silk cocoons may comprise the selective removal of sericin from the silk or silk cocoons and may use a proteolytic enzyme which cleaves sericin, but produces little or no cleavage of fibroin. The proteolytic enzyme may comprise trypsin. Alternatively, the proteolytic enzyme may comprise proline endopeptidase. Degumming may use an enzyme solution in a buffer containing ammonium hydroxide.

Degumming may be performed at any suitable temperature, for example, a temperature of less than 100° C. Preferably, degumming is performed at a temperature in the range of approximately 20° C. to approximately 40° C. Good results have been observed where degumming is performed at a temperature of approximately 37° C.

The chaotropic agent may be removed by dialysis to provide a regenerated fibroin solution. For example, dialysis may be performed using high grade deionised grade II water and is typically carried out using ultrapure grade I water ultrapure water.

Dialysis may be performed at any suitable temperature, for example within a temperature range of approximately 0° C. to approximately 40° C. More preferably, dialysis may be performed in a temperature range of approximately 2° C. to approximately 10° C. Good results have been achieved at a temperature of approximately 4° C. to 5° C.

The method may comprise the step of concentrating the regenerated fibroin solution. The solution may be concentrated by exposing sealed dialysis tubes, or other dialysis vessel to a vacuum. The regenerated fibroin solution may be concentrated to a concentration of approximately 5-25% w/v. Preferably, the regenerated fibroin solution is concentrated to a concentration of approximately 8-22% w/v. More preferably, the regenerated fibroin solution is concentrated to a concentration of approximately 8-12% w/v. By way of example, particularly good results have been achieved where the regenerated fibroin solution is concentrated to a concentration of approximately 10% w/v.

Preferably, the dialysis tubes, or other vessel is removed before the gel is frozen.

According to a second aspect of the present invention there is provided a method for the preparation of an implantable material for the repair, augmentation or replacement of bone from a fibroin solution, the method comprising the steps of:
preparing a gel from the fibroin solution;
preparing a material by subjecting the gel to one or more steps of freezing and thawing the gel,

wherein the method comprises the further step of subsequently treating the material with an isocyanate.

The gel may be treated with phosphate ions.

Most preferably, the step of preparing the gel from the fibroin solution is performed in the presence of phosphate ions.

It will be appreciated that the preferred features described in relation to the first aspect of the invention may apply to the second aspect of the invention.

According to a third aspect of the invention, there is provided a method for the preparation of an implantable material for the repair, augmentation or replacement of bone from a regenerated fibroin solution, wherein the regenerated fibroin solution is prepared by a method comprising step of treating silk or silk cocoons with an ionic reagent comprising an aqueous solution of monovalent cations and monovalent anions, the cations and anions having ionic radii of at least 1.05 Angstroms and a Jones-Dole B coefficient of between −0.001 and −0.05 at 25° C.

It will be appreciated that the preferred features described in relation to the first and second aspects of the invention may apply to the third aspect of the invention.

According to a fourth aspect or the present invention there is provided an implantable material obtainable by any one of the methods described herein.

According to a fifth aspect or the present invention there is provided an implantable fibroin material for use as a bone repair, augmentation, or replacement material, wherein the material has the following properties:
a compressive toughness of between approximately 1 kJ m⁻³ and approximately 20 kJ m³ at 6% strain measured by the J-integral method;
a compressive strength of between approximately 0.1 MPa and approximately 20 MPa at 5% strain; and
a mean compressive elastic modulus of between approximately 100 MPa and approximately 500 MPa at 5% strain.

The material may comprise a compressive toughness of approximately 1 kJ m⁻³ to approximately 5 kJ m⁻³ at 6% strain. Preferably, the material comprises a compressive toughness of approximately 1.3 kJ m⁻³, which is the approximate compressive toughness of healthy bone.

The ultimate compressive strength of the material may depend upon the target site of implantation. For example, if the material is for placement next to osteoporotic cancellous bone, to avoid high stress accumulation and stress shielding, the material may comprise a compressive strength (stress to yield point) of approximately 0.1 MPa to approximately 2 MPa. If the material is intended for placement next to healthy cancellous bone, the material may comprise an ultimate compressive strength (stress to yield point) of approximately 5 MPa. Alternatively, if the material is intended for placement next to cortical bone, the material may comprise an ultimate compressive strength (stress to yield point) of at least 10 MPa.

Preferably, the material comprises an ultimate compressive strength (stress to yield point) of approximately 5 MPa to approximately 14 MPa. Preferably, the material comprises an ultimate compressive strength (stress to yield point) of at least 12 MPa. Most preferably, the material comprises an ultimate compressive strength (stress to yield point) of approximately 14 MPa.

The material may comprise a compressive elastic modulus of between approximately 100 MPa and approximately 400 MPa at 5% strain. Most preferably, the material comprises a compressive elastic modulus of approximately 175 MPa at 5% strain.

The material may comprise a fibroin-apatite. Preferably, the apatite is distributed throughout the material as a fibroin-apatite nanocomposite. This can be achieved by preparing the gel from the fibroin solution in the presence of phosphate ions. The fibroin-apatite nanocomposite may comprise one or a combination of fibroin-hydroxyapatite and fibroin-chlorapatite.

Additionally, or alternatively, the apatite may be present as a coating on the surface of the fibroin material, which is achieved by preparing the gel from the fibroin solution and then subsequently exposing the gel to phosphate ions.

Most preferably, the apatite is present as both a nanocomposite dispersed throughout the material and a coating on the surface of the material.

Preferably, the material further comprises intercommunicating pores. The pores may cover from approximately 10% up to approximately 80% of a cross-section of the material. In a preferred embodiment, the pores cover approximately 75% of a cross-section of the material.

The pores may range from approximately 10 μm to approximately 1000 μm in diameter. The average pore diameter may range from approximately 25 μm to approximately 400 μm. Preferably, the mean pore diameter is between approximately 100 μm and approximately 300 μm.

Preferably, at least part of the apatite is present within walls of the pores.

The material may comprise a calcium phosphate content of between approximately 15% w/v and approximately 70% w/v. Preferably, the material comprises a calcium phosphate content of approximately 30% w/v.

The material may comprise covalent fibroin-fibroin cross-links. The amount of cross-linking may be tuned according to the intended application of the material, for example, by increasing the stiffness or decreasing the resorbability of the material by increasing the amount of cross-linking in the material.

The material may be biocompatible and at least partially bioresorbable. The material may have a resorption half-life of approximately 6 months to approximately 12 months. Preferably, the material has a resorption half-life of approximately 9 months. The material may be completely resorbed in approximately 12 months to approximately 24 months. Preferably, the material is completely resorbed in approximately 12 months.

Preferably, the material elicits a negligible or no immune response when implanted in an organism. Preferably, the material has negligible pyrogen content.

Preferably, the material is osteogenic and shows new bone formation after implantation in vivo. The material may show new bone formation within 6 months of implantation in vivo. Preferably, the material shows new bone formation within 8 weeks of implantation in vivo.

The material may comprise a rough adherent surface for the binding of a fibrin matrix to facilitate the migration of osteogenic cells to the surface of the material.

The material may be seeded with tissue cultured cells including bone marrow stromal cells, mesenchymal stem cells, cells from an osteogenic cell line, blood cells, or cells harvested from a target patient.

According to a sixth aspect or the present invention there is provided an implant for the repair, augmentation, or replacement of substantially all or part of one or more bones, or as a substitute for bone grafts in orthopaedic applications comprising an implantable material as described herein.

The implant may comprise a bone anchor embedded in the material. The bone anchor may comprise a plurality of threads or filaments embedded in the material.

According to a seventh aspect of the invention there is provided a use of an implantable material as described herein for the repair, augmentation or replacement of substantially all or part of one or more bones, or as a substitute for bone grafts, or as a securing device in orthopaedic applications.

Other objects, features and advantages of the invention will be apparent from the following detailed disclosure, taken in conjunction with the accompanying figures.

### BRIEF DESCRIPTION OF THE DRAWINGS

The invention will now be described further by way of example only and with reference to the accompanying drawings in which:

FIG. 1 a scanning electron micrograph (SEM) image of a cross-section of a porous implantable bone repair material according to the invention;

FIG. 2 an energy dispersive X-ray (EDX) spectrum showing the calcium phosphate content of the material shown in FIG. 1;

FIG. 3 a high magnification SEM image of a scaffold pore wall of the porous implantable bone repair material shown in FIG. 1;

FIG. 4 a scanning electron micrograph (SEM) image of a cross-section of a porous implantable bone repair material according to the invention;

FIG. 5 a scanning electron micrograph (SEM) calcium map of the porous implantable bone repair material shown in FIG. 4;

FIG. 6 a scanning electron micrograph (SEM) phosphate map of the porous implantable bone repair material shown in FIG. 4;

FIG. 7 an energy dispersive X-ray (EDX) spectrum showing the calcium phosphate content of the porous implantable bone repair material shown in FIG. 4;

FIG. 8 a fourier transform infra-red spectrum showing stretching/bending modes of a porous implantable bone repair material according to the invention;

FIG. 9 a powder X-ray diffraction pattern of a porous implantable bone repair material according to the invention;

FIG. 10 a plot showing compressive stress (MPa) against compressive strain for a control sample of a porous sintered ceramic calcium phosphate bone repair material from Endobon® (A) and three samples of a porous implantable bone repair material according to the invention each with a 30 wt % mineral content (B-D);

FIG. 11 a bar graph showing the IL-1β response from human blood (pg mL-1) to a control sample of an E. coli lipopolysacharide and a porous implantable bone repair material according to the invention;

FIG. 12 a haematoxylin and eosin stained glycol methacrylate resin section of a porous implantable bone repair material according to the invention seeded with bone marrow stromal cells at eight weeks post-implantation in immuno-compromised mice;

FIG. 13 a high magnification image of a section of the porous implantable bone repair material according to FIG. 12 showing palisades of osteoblasts (arrows) on the osteoid surface and loose connective tissue (CT);

FIG. 14 a high magnification image of a section of the porous implantable bone repair material according to the invention showing osteoclastic remodelling (arrows) and newly formed bone; and

FIG. 15 a high magnification image of a section of the porous implantable bone repair material according to FIG. 14 showing osteoclastic infiltration and remodelling of the newly synthesised bone by multinucleate osteoclasts (arrows).

### DETAILED DESCRIPTION OF THE INVENTION

An implantable material for the repair, augmentation or replacement of bone according to the invention comprises fibroin. The material has load-bearing capacity comprising compressive strength and compressive toughness approximately matching that of bone at the site of implantation to enable it to maintain its mechanical integrity without undue distortion when subjected to the forces applied to it by normal physical activity.

The fibroin can be prepared from a Mulberry silk, a Wild Silk, a recombinant silk or a combination of these silks.

### Load-Bearing Properties

The compressive strength, compressive toughness and compressive elastic modulus values of the material approximate to those of healthy human bone and enable immediate load-bearing. The load-bearing properties also prevent unwanted resorption of adjacent bone resulting from high local stress concentration or stress-shielding.

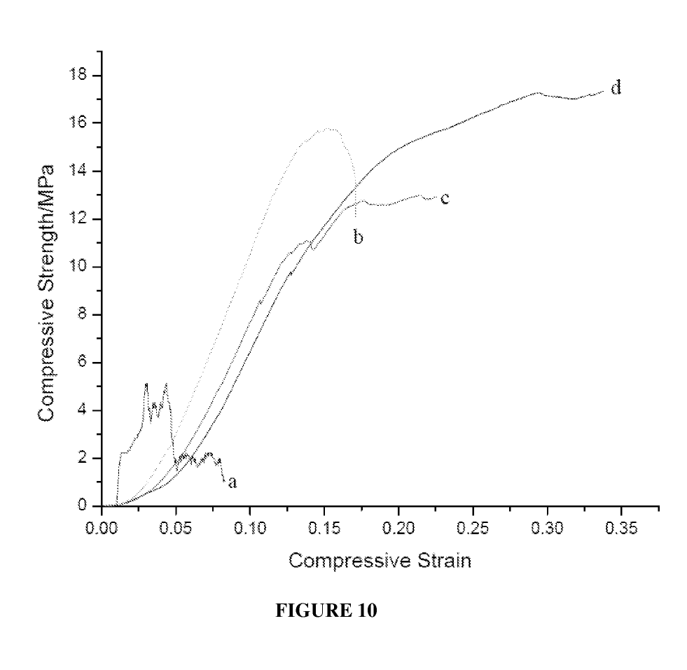

Compressive strength is the capacity of a material to withstand axially directed pushing forces. By definition, the compressive strength of a material is that value of uniaxial compressive stress reached when the material fails completely. A stress-strain curve is a graphical representation of the relationship between stress derived from measuring the load applied on the sample (measured in MPa) and strain derived from measuring the compression of the sample. As can be seen from FIG. 10, when a sample of the material is tested wet the material has an unconfined ultimate compressive strength (stress to yield point) of up to 14 MPa (n=5).

Compressive toughness is the capacity of a material to resist fracture when subjected to axially directed pushing forces. By definition, the compressive toughness of a material is the ability to absorb mechanical (or kinetic) energy up to the point of failure. Toughness is measured in units of joules per cubic metre (J m⁻³) and can be measured as the area under a stress-strain curve. Therefore, as can be calculated from FIG. 10, when a sample of the material is tested wet the material has a mean unconfined compressive toughness of up to 11.93±8.40 kJ m⁻³, n=6 (obtained using the J-integral method).

Compressive elastic modulus is the mathematical description of the tendency of a material to be deformed elastically (i.e. non-permanently) when a force is applied to it. The Young's modulus (E) describes tensile elasticity, or the tendency of a material to deform along an axis when opposing forces are applied along that axis; it is defined as the ratio of tensile stress to tensile strain (measured in MPa) and is otherwise known as a measure of stiffness of the material. The elastic modulus of an object is defined as the slope of the stress-strain curve in the elastic deformation region. The compressive elastic modulus can be calculated from FIG. 10, which shows when a sample of the material is tested wet the material has an unconfined compressive elastic modulus of 175 MPa (n=5). Covalent cross-linking of the fibroin allows the stiffness of the material to be controlled. With a di-isocyanate cross-linking agent, the density of covalent cross-linking in the fibroin can be tuned by varying the exposure time of the material to the agent to vary the stiffness of the material.

As can be seen from FIG. 10, the compressive strength, compressive toughness and compressive elastic modulus (measured in the elastic deformation phase) of the implantable bone repair material (samples B, C and D) are considerably higher than a tested porous sintered ceramic calcium phosphate bone repair material known as Endobon®, manufactured by BIOMET Orthopaedics Switzerland GmbH (sample A). The variation in the samples B, C and D is thought to be largely due to difficulty in preparing smooth, exactly parallel faces on the samples taken.

### Mineralisation

Mineral density is a major determinant of compressive strength and compressive elastic modulus in mineralized composite materials. Therefore, mineralisation has an impact on the load-bearing properties of the material.

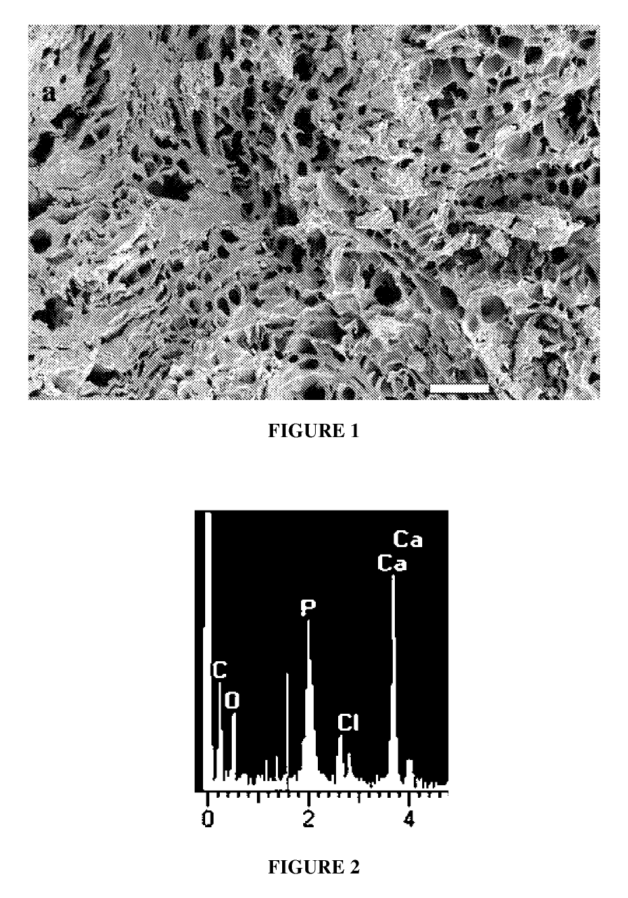

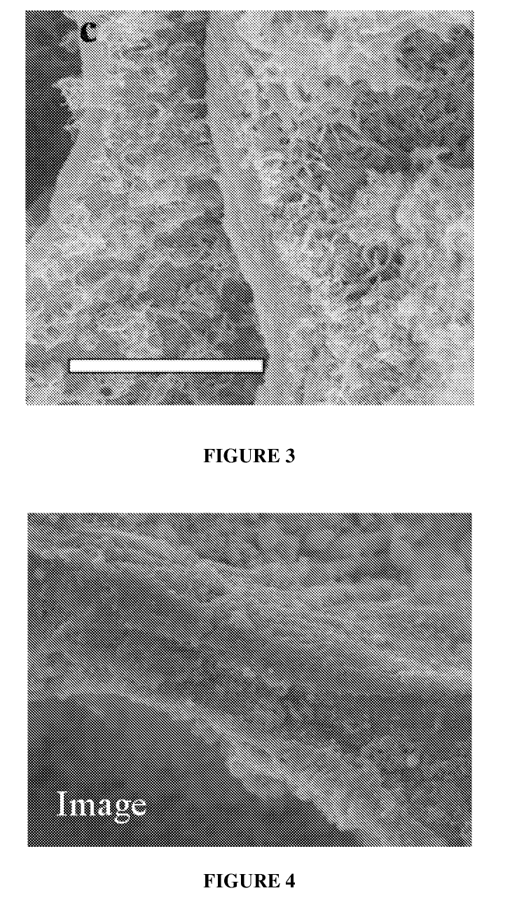

Referring to FIGS. 1-3, a sample of the material according to the invention shows a porous mineralized architecture with a high content of calcium phosphate crystallites. FIG. 3 shows that walls of the pores have crystallites that practically cover the surface of the wall and extend right up to the two fractured surfaces of those walls, which indicates that the crystallites adhere tightly to the pore walls.

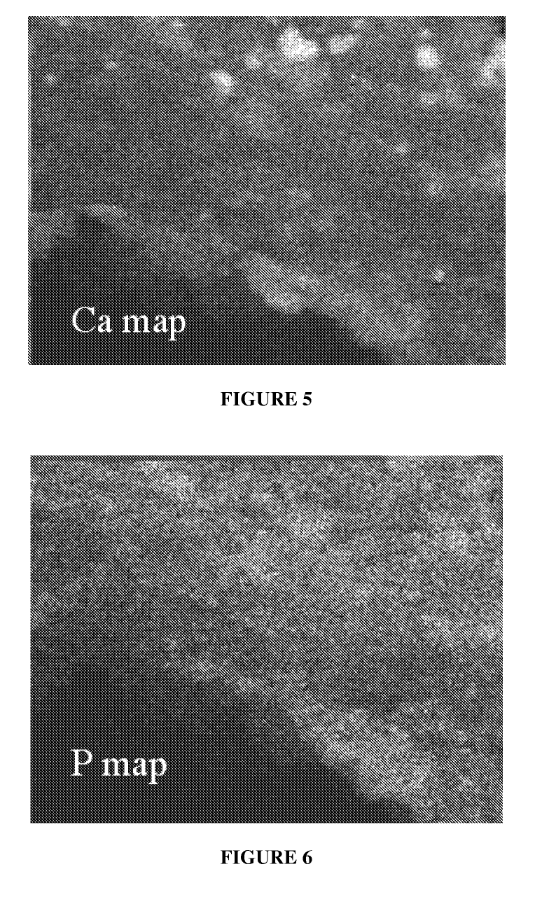

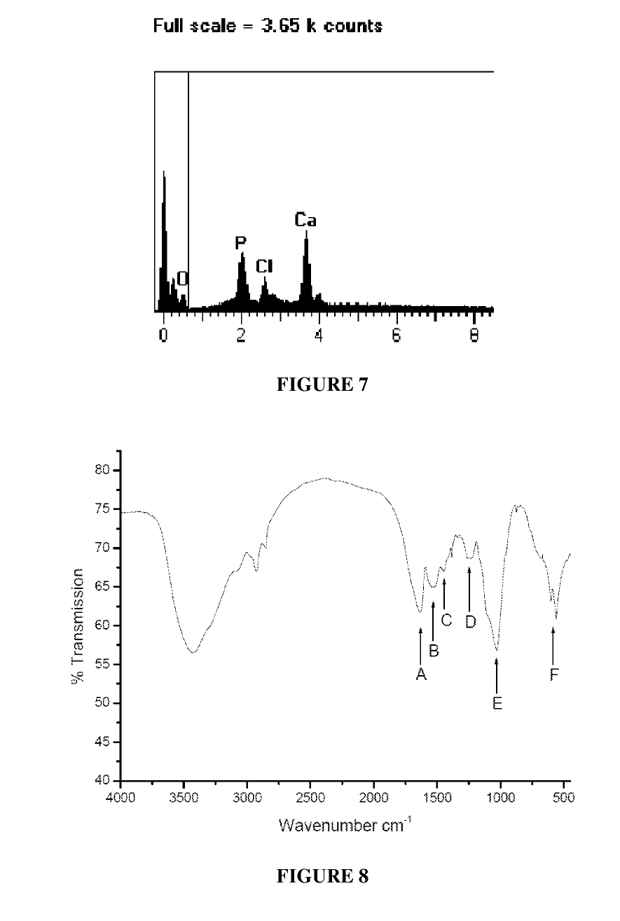

FIGS. 5 and 6 show calcium and phosphate maps of a further sample of the material according to the invention. The energy dispersive X-ray (EDX) spectrum of FIG. 7 shows the calcium phosphate content of the same sample of the material. The material shown in FIGS. 5-7 can be seen to largely comprise fibroin and apatite (a calcium phosphate ceramic). The fibroin-apatite composite is in part, a true apatite-protein nanocomposite like natural bone and not just fibroin coated with apatite although some of the apatite is indeed present as a firmly adherent coating on the fibroin.

### Osteogenic Properties

Osteogenesis is the process of laying down new bone material using osteoblasts. Osteoblasts build bone by producing osteoid to form an osteoid matrix, which is composed mainly of Type I collagen. Osseous tissue comprises the osteoid matrix and minerals (mostly with calcium phosphate) that form the chemical arrangement termed calcium hydroxyapatite. Osteoblasts are typically responsible for mineralization of the osteoid matrix to form osseous tissue. The osteoconductivity and osteoinductivity of the material has an impact on osteogenesis.

### Osteoconductivity

Osteoconductivity is generally defined as the ability of a material to facilitate the migration of osteogenic cells to the surfaces of a scaffold through the fibrin clot established immediately after implantation the material. The porosity of a material affects the osteoconductivity of that material.

The scanning electron micrograph (SEM) image in FIG. 1 (scale bar=200 μm) shows that the material according to the invention comprises a porous mineralized architecture when a cross-section of a sample of the material is taken. The material comprises interconnected pores. Furthermore, FIG. 3 (scale bar=10 μm) shows a pore wall of the same sample of the material.

### Osteoinductivity

Osteoinductivity is defined as the ability of the material to promote differentiation of the osteoprogenitor cells (osteoblasts), which is a component of osseous (bone) tissue. The mineralization and the addition of growth factors affects the osteoinductivity of a material.

The material according to the invention is highly osteogenic and shows evidence in vivo within 8 weeks of implantation of the laying down and remodeling of bone (FIGS. 12-15). In this respect, FIGS. 12 to 15 show haematoxylin and eosin stained glycol methacrylate resin sections of the material that were seeded with bone marrow stromal cells at the eight week post-implantation stage in immuno-compromised mice.

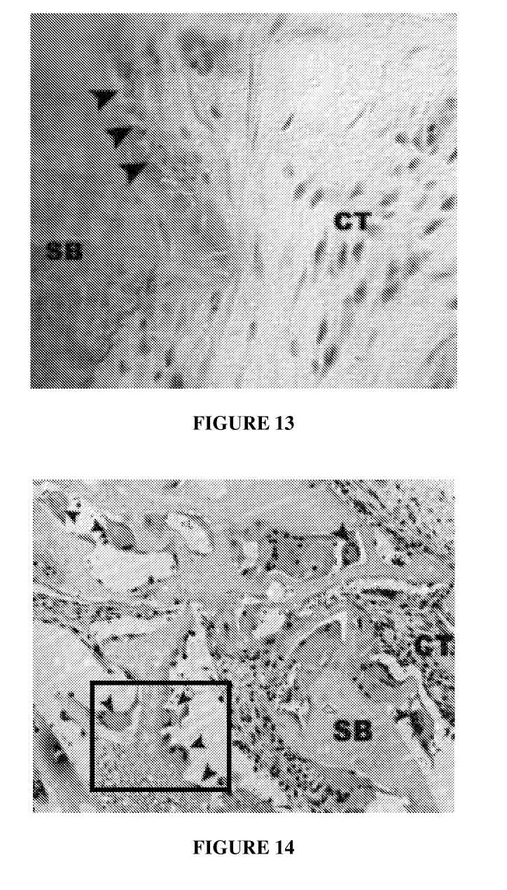

FIG. 12 (scale bar=100 μm) shows formation of a new osteoid matrix (arrows) which has been secreted by osteoblasts on the surface of the material (SB). FIG. 13 (scale bar=20 μm) shows a magnified portion of the osteoid matrix seen in FIG. 12, in which palisades of osteoblasts (arrows) can be seen on the osteoid surface and loose connective tissue (CT).

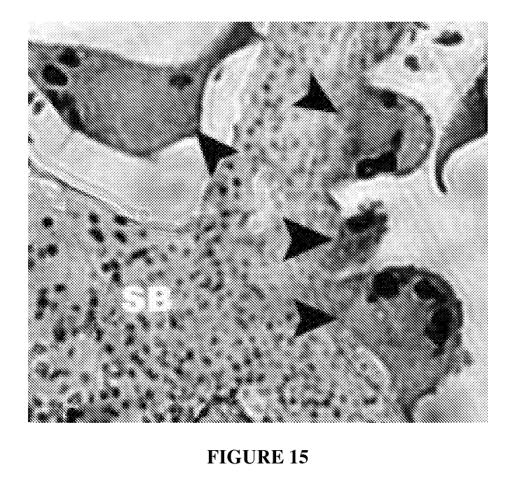

FIG. 14 (scale bar=100 μm) shows osteoclastic remodelling (arrows) of the material and newly formed bone can be seen in the implant. FIG. 15 (scale bar=20 μm) shows a magnified portion of a section of FIG. 14, in which osteoclastic infiltration and remodelling of the newly synthesised bone by multinucleate osteoclasts (arrows) can be seen. The material can also comprise additional resorbable biopolymers, drugs, growth factors, filler particles and minerals.

### Resorbability

Resorbability is the ability of the material to be broken down. The aim for a BRM is that the material is gradually broken down to allow it to be replaced by endogeneous bone tissue.

The material according to the invention demonstrates a slow resorbability, showing a halving of the unconfined compressive elastic modulus within 12 weeks to 9 months, depending on the extent of the introduced cross-linking. The material shows evidence in vivo within 8 weeks of implantation of resorption of the fibroin (FIGS. 14 and 15).

Covalent cross-linking of the fibroin allows the resorbability of the material to be controlled. In particular, cross-linking of the fibroin renders the fibroin less hydrophilic and more resistant to enzymatic attack, which increases the resorption time. With a di-isocyanate cross-linking agent, the density of covalent cross-linking in the fibroin can be controlled to vary the hydrophobicity and resorbability of the material.

When a calcium chloride agent is used to introduce calcium into the material, the material shows some chloride substitution of the apatite to form a material which is part chlorapatite and part hydroxyapatite. The chloride substitution is thought to speed up resorption of the apatite compared with unsubstituted hydroxyapatite.

### Use of the Material

The material can be trimmed with a sharp scalpel and can be cast in a mould and or machined into rods or prisms or into any three dimensional shape to mimic that of the bone or part of the bone to be replaced. It can be readily formed into pieces with average dimensions of 1 to 50 mm for use in impaction grafting or for placing between fractured or fragmented bones. It can be readily drilled and held in place by resorbable or nonresorbable screws, pins, or plates. Furthermore, it can be held in place by an anchor of threaded, braided or twised fibres or threads, or a cable embedded in the material.

The material could also be cast, milled or otherwise shaped to form a securing device, such as a screw or pin to secure implants to existing bone.

### Overview of the Method for Making Implantable Bone Repair Material

The implantable material is prepared by an optimized method as described below.

Silk or silk cocoons are treated with ammonia or with an aqueous solution containing ammonium ions.

The silk or silk cocoons are degummed under mild conditions by selectively removing the sericin. This is done by enzymatically cutting and removing the sericin using a suitable enzyme which cleaves sericin, but produces little or no cleavage of fibroin.

The silk or silk cocoons are dried by extracting water.

The silk or silk cocoons are dissolved in an aqueous lithium bromide solution at one or more of a temperature of less than 60° C. and/or with a concentration of lithium bromide solution of less than 9.5M and/or for a period of time of less than 24 hours.

The chaotropic agents are removed by dialysis using ultrapure water in the cold at a temperature of approximately 4-5° C. The resulting solution is concentrated to provide an optimized regenerated fibroin solution.

The fibroin solution can be concentrated.

The solution is transferred to a mould for gelling, or alternatively, the solution is left in the dialysis vessel. The solution is gelled whilst introducing phosphate ions into the fibroin solution by treating the solution with a concentrated buffered solution containing phosphate ions. In the preferred embodiment, the buffered phosphate solution comprises dihydrogen sodium phosphate buffered with 2-amino-2-(hydroxymethyl)propane-1,3-diol (Tris) buffer, adjusted to an alkaline pH.

The gel is removed from the mould or dialysis vessel prior to freezing.

The gel is subjected to one or more freezing cycles. Each freezing cycle comprises a freezing step and a thawing step. By freezing the gel the water droplets are turned to ice crystals which form pockets or pores within the gel. Therefore, subjecting the gel to one or more freezing cycles introduces intercommunicating pores.

The fibroin gel is treated with a concentrated buffered solution containing calcium ions to form a fibroin-apatite material. The apatite is present as a nanocomposite in and on the walls of the pores. The buffered calcium solution comprises calcium chloride also buffered with Tris to an alkaline pH.

The material is washed in an aqueous solution of ethanol to remove excess salt and to facilitate the formation of the silk II (beta sheet) form of the fibroin.

As much free water as possible is removed from the material, by for example, vacuum drying.

The fibroin in the material is optionally cross-linked using an undiluted isocyanate or a highly concentrated isocyanate solution in dimethylsulphoxide or other organic solvent. Excess isocyanate is removed by treating the material with a dry solvent.

The resultant material is used as an implantable material for the repair, augmentation or replacement of bone.

Treatment with Ammonia, or Ammonium Ions

It was discovered that treatment of the silk with ammonia gas, or a dilute solution of ammonia or an ammonium salt greatly increased the readiness of silk to dissolve in a lithium bromide solution or other chaotropic agent. In this step, it is believed that ammonium ions act as a ‘salting in’ reagent, which increases the subsequent solubility of the protein in the chaotropic reagent by assisting in the removal of an inner water shell surrounding the protein chains and by binding to the charged amino acid side chains of the fibroin.

It was found that this treatment was effective when applied at one or all of three stages: directly to undegummed cocoons; to raw silk fibres, to degummed or partially degummed silk whether degummed by conventional industrial degumming methods or by enzymatic degumming. Ammonia or ammonium ions were also effective when included as a component of the buffer used for enzymic degumming. Thus any of these methods of treatment of silk with ammonia or ammonium ions could be used to reduce the temperature, or the time, or the concentration of the chaotropic agent required to dissolve the silk resulting in reduced damage to the fibroin and a saving in process costs.

Treating B. mori silk with ammonia or ammonium ions enabled the time for dissolving the silk in 9.3 M lithium bromide solution at 60° C., to be cut from several hours to 15 minutes. Alternatively, ammonia or ammonium ion treatment enabled 7M lithium bromide to be used in place of 9.3 M at 60° C. It also enabled the silk to be completely dissolved in 9.3M lithium bromide solution at 20° C. within 24 hours. It further enabled the silk to be completely dissolved in 9.3M lithium bromide at 37° C. within 4 to 5 hours.

Therefore, it was found that treatment with ammonia or ammonium enables a range of milder treatments in which the temperature, concentration of the chaotropic agent or time required for solution can be varied singly or in combination. These milder treatments resulted in more rapid gelling times for the fibroin solution and stronger stiffer materials at the end of the process.

It is currently considered that other pairs of ions with the same size, for example, potassium chloride will also have the same effect and could be used in place of the ammonia. This is supported by two lines of evidence: (1) The Jones-Dole viscosity (a measure of the chaotropicity) of potassium and chloride ions are similar as is the charge density enabling the ions to form ion pairs and help to remove an inner water shell of the protein (properties shared with ammonium chloride; and (2) Potassium chloride has been used to “salt in” proteins at salt concentrations generally ranging from 50 mM to 600 mM.

Furthermore, certain other ionic reagents comprising an aqueous solution of monovalent cations and monovalent anions could provide the same effect. Particularly, it is thought that an ionic reagent comprising monovalent cations and monovalent anions having ionic radii of at least 1.05 Angstroms and a Jones-Dole B coefficient of between −0.001 and −0.05 at 25° C., would provide the same effect as that described in relation to the ammonium ions.

Suitable ionic reagents may include aqueous solutions of ammonium hydroxide, ammonium chloride, ammonium bromide, ammonium nitrate, potassium hydroxide, potassium chloride, potassium bromide, potassium nitrate, rubidium hydroxide, rubidium chloride, rubidium bromide and rubidium nitrate.

### Degumming

The choice of the degumming method was also found to be crucial for the gelling time of the fibroin and stiffness and strength of the final material. Commercial reeling and degumming processes both use temperatures of around 100° C. and the use of sodium carbonate and/or Marseille's soap and it was found that reeled raw silks and degummed silks dissolved less readily than cocoon silks probably as a consequence of this treatment.

Degumming with commercial alcalase (bacterial subtilisin) enabled the degumming temperature to be reduced to 60° C. Alcalase is a member of the Serine S8 endoproteinase family and is likely to degrade fibroins badly as it has a broad specificity with a preference for a large uncharged residue in the P1 position. B. mori and Antheraea pernyi heavy chain fibroins have many predicted cleavage sites for this enzyme. The susceptibility of B. mori fibroin to alcalase cleavage was confirmed by polyacrylamide gel electrophoresis of a regenerated fibroin solution prepared from alcalase degummed silk.

In the case of degumming using trypsin the temperature for degumming could be reduced to 20° C. to 40° C. and gave gels with reduced gelling times, and with improved stiffness and strength compared with conventional high temperature degumming procedures. In contrast to alcalase, the tool PeptideCleaver showed few predicted trypsin cleavage sites in the consensus sequence of the repetitive crystalline domains and of the hydrophilic spacers of B. mori fibroin heavy chain fibroin and none in the consensus sequence or hydrophilic spacer in A. pernyi heavy chain fibroin. This suggested that it might be beneficial to degum silks in trypsin for the preparation of regenerated fibroinsolutions. Trypsin was indeed found to be highly advantageous for degumming silk for the formation of improved regenerated fibroin solutions.

Silks degummed with trypsin gave regenerated silk solutions with shorter gelation times and capable of forming stiffer gels than those obtained from regenerated silk prepared from silk degummed with alcalase. Degumming with trypsin gave gelling times of less than 5 minutes on exposure to one gelling agent, glacial acetic acid vapour and also gave the stiffest and strongest materials suggesting that trypsin under these conditions produced much less chain cleavage than alcalase treatment.

It will be understood that other proteolytic enzymes producing little or no cleavage of fibroin may also be advantageous for degumming silks for the preparation of improved regenerated fibroin solutions. The observation that B. mori heavy chain fibroin contains very little proline while this amino acid is relatively abundant in sericin suggested that proline endopeptidase would be an ideal candidate to selectively remove sericin while producing little or no damage to fibroin.

### Drying

The silk or silk cocoons are air dried overnight at room temperature in less than 20% humidity and in the presence of anhydrous calcium chloride.

The removal of substantially all of the water through drying increased the concentration of the ions in the solution, which was thought to enhance the effects of the ions and the resultant material.

Other known methods of drying such as freeze drying and drying through the application of heat would achieve the same effect. If heat drying is used, a temperature of less than 100° C. is thought to result in an improved fibroin material.

### Dialysis

It was found to be highly beneficial to dialyse regenerated fibroin solutions against type I milliQ™ water (available from Millipore™, 290 Concord Road, Billerica, Mass. 01821, US), otherwise known as ultrapure water, to remove the chaotropic agent from the silk solution.

It was noted that PIPES or Tris buffers or impurities in deionised water adversely affected the stiffness and strength of the final product when used as dialysants. It was noted that the inclusion of PIPES or Tris buffers or impurities in the dialysant also increased the viscosity of the regenerated silk solution, probably as a result of their ability to encourage the aggregation of the fibroin chains by binding to them. This is thought to be disadvantageous in the formation of strong and stiff fibroin gels.

It is considered that it may be of further advantage to use cocoon or raw silks degummed with trypsin in ammonium carbonate buffer at 40° C.

### Preparation of a Gel

The optimised regenerated fibroin solution was gelled by exposure to an aqueous buffered solution, containing dihydrogen sodium phosphate. The concentration of the dihydrogen sodium phosphate was 0.9 M in 1% Tris buffer and adjusted to pH 9.0. The concentration of the dihydrogen sodium phosphate and the length of exposure of the material to it were crucial to the pore size and the strength and stiffness of the resulting gel. It was discovered that by gelling the solution in the presence of phosphate ions allowed the phosphate ions to disperse throughout the solution and therefore, be integrated into the gel. This facilitates the formation of the fibroin-apatite nanocomposite when calcium ions are added at a later stage. It was found that if the gel was subsequently treated with phosphate ions, an apatite coating was achieved when calcium ions were added at a later stage.

Furthermore, it was found that freezing under-gelled fibroin resulted in a reduction in the pore size and a weaker material while strong over-gelation gave non-porous gels containing a low density of large splits produced by large ice crystals. It was found that the length of exposure and concentration of the buffer or vapour required for optimal gelation depended on the geometry and size of the fibroin cast. Thus longer treatments were required to optimally gel fibroin in moulds constructed from 20 mm diameter dialysis tubing compared with 10 mm dialysis tubes.

It was found to be advantageous to gel 10% w/v optimised regenerated fibroin solution prepared from trypsin degummed silk contained in 20 mm diameter dialysis bags for 2 hours at 4° C.

Although the preferred embodiment combines introducing phosphate ions and gelling the fibroin solution in a single step, other gelling agents or methods can be used to gel the fibroin solution before introducing the phosphate ions, including by way of example only, heat, microwave radiation, ultrasound treatment, laser radiation, acidic solutions and acidic vapours.

### Freezing

For the preparation of porous implantable material the gel can be rendered porous by freezing. Freezing is thought to result in phase separation of a fibroin-rich phase from a fibroin-poor phase and ice crystal formation in the latter. These two mechanisms are thought to combine to give rise to a high density of interconnected pores in the gel.

It was found that removal of the dialysis vessel or mould gave a greater degree of porosity and intercommunicating pores.

The freezing step also makes the fibroin in the pore walls insoluble in water and most other aqueous solvents suggesting that it has been partially converted to the insoluble silk II state in which intra- and inter-molecularly bonded beta-sheets predominate. This transition to the silk II state may result from the removal of water from the protein chains produced by a combination of phase separation and their alignment and pulling together, both as a consequence of ice crystal formation. Thus the formation of the insoluble silk II state rather closely mimics the natural process by which silks are extruded, from the silk worm which also depends on phase separation, loss of water from the fibroin-rich phase and strain dependent orientation and silk II formation.

For a single freezing cycle, the temperature of the freezing step has a small effect on the pore size with the largest pores produced by freezing between −12° C. to −16° C. Varying the temperature and including low concentrations of antifreezes or sugars in the regenerated protein solution can be used to vary the ice crystal size and morphology and hence the size and shape of the pores in the material.

Increasing the number of freezing cycles produced an increase in the size of the pores as a result of damage by ice crystals. This was accompanied by some loss in the stiffness and strength of the final material.

It will be understood that methods other than gelation and freezing can be used to introduce intercommunicating pores into the optimised regenerated fibroin solution. By way of example only these include salt leaching and gas foaming.

### Introducing Calcium Ions

The calcium ions form a apatite with the phosphate ions. If phosphate ions are dispersed throughout the fibroin solution prior to gelling, then a fibroin-apatite nanocomposite is achieved. However, if the fibroin solution is first gelled and then treated with phosphate ions, an apatite coating is observed, but not a nanocomposite.

The use of calcium chloride induces some chloride substitution of the apatite. This is desirable as it is thought to speed resorption of the apatite compared with unsubstituted hydroxyapatite. A further embodiment uses calcium nitrate solution in place of calcium chloride solution, which avoids the presence of chloride ions in the apatite and results in the formation of a pure hydroxyapatite rather than a partially chloride-substituted hydroxyapatite (i.e. a part chlorapatite, part hydroxyapatite).

The material is treated with calcium ions at a basic pH, which avoids the formation of an acidic or amorphous apatite. Good results have been achieved when the material is treated with calcium ions at a pH of approximately 9.0.

Other elements can be incorporated into the fibroin in the fibroin solution before conversion of the gel to a fibroin-apatite material. These include by way of example only short staple fibres, filler particles, bone promoting factors and drugs, antineoplastic drugs, antibiotics, other biopolymers and other active principles.

A final concentration of 30% mineral by dry weight in the implantable bone repair material is preferable, which is obtained by using a buffered 0.9 M dihydrogen sodium phosphate solution and a buffered 1.5 M calcium chloride solution. Higher mineral contents up to 70% in the implantable bone repair material can be obtained by increasing the phosphate and calcium ions concentrations in the phosphate- and calcium-ion solutions stoichiometrically. However implantable bone repair materials containing more than a 40% mineral content were found to be more brittle than those containing a 30% mineral content.

Treatment with Ethanol Solution

Treating the material with an aqueous ethanol solution after freezing is thought to facilitate the formation of the silk II (beta sheet) inter- and intra-molecular hydrogen bonds, which improve the mechanical stability of the gel and increase insolubility and resistance to enzymatic attack.

### Further Drying

The material is, for example, brought to dry ethanol over 2 days and vacuum dried at 40° C. to remove substantially all, if not all, free water.

Other known methods of drying such as freeze drying and drying through the application of heat would achieve the same effect. If heat drying is used, a temperature of less that 100° C. is thought to result in an improved material.

### Cross-Linking

The fibroin-apatite is cross-linked.

In a preferred embodiment, the fibroin-apatite is cross-linked with an undiluted isocyanate, such as hexamethylene di-isocyanate, in the absence of water or other swelling agents. This step increases the stiffness of the implantable bone repair material and increases the resistance of the implantable bone repair material to enzymatic attack thereby slowing resorption.

It was found that if a swelling agent was used, this caused the fibroin to swell, which resulted in a separation of the apatite from the fibroin. Consequently, this caused the material to have reduced stiffness, which is turn resulted in a tendency of the material to flex and cause the apatite to ‘flake’ out of the material.

It was also found that varying the length of exposure of the fibroin-apatite to an isocyanate cross-linking agent could be used to tune the density of covalent cross-linking and hence the stiffness of the implantable bone repair material.

It was also found that varying the density of covalent cross-linking could be used to vary the resistance of the fibroin gel to enzymatic attack and thereby extend the resorption time in a controlled way. Attempts to cross-link the fibroin in the material with solutions of 20% hexamethylene di-isocyanate in dimethylsulphoxide (DMSO) using the published protocol described by Arai, T, Ishikawa, H., Freddi, Winkler, G S and Tsukada, M (2001) op.cit., did not produce satisfactory implantable bone repair material. In the published protocol, it is through that, because swelling of the fibroin-apatite in the DMSO resulted in a separation of the mineral from the fibroin.

Therefore, the method uses an isocyanate cross-linking agent in the absence of water or other swelling agents such as dimethylsulphoxide. Isocyanate cross-linking does not appear to interfere with the biocompatibility of the material provided that excess cross-linking agent is removed by thorough washing. This was established in vitro by growing human stromal cells on and in the porous fibroin-apatite composite and in vivo after subcutaneous implantation into mice (see protocols below).

It will be appreciated that other cross-linking agents could be used.

### Overview of the Method of Implantation of the Implantable Bone Repair Material

In a preferred embodiment, the material is implanted directly into the bone without it first being seeded with tissue cultured cells.

Alternatively the material can be seeded immediately prior to implantation with tissue cultured cells or blood cells or cells harvested from the patient shortly before implantation of the material.

By way of example only, such tissue cultured cells include bone marrow stromal cells, or mesenchymal stem cells, or an osteogenic cell line.

As a further alternative, the material can be seeded with cells and then subjected to tissue culture with or without applied cyclical strain, to accelerate the formation of bone in the material before implantation.

The size and shape of the material can be varied for different applications in bone repair. Thus anatomically-shaped monoliths can be produced by casting the material in a suitably shaped mould or by grinding, cutting or otherwise machining a larger block of material. Alternatively, cylindrical rods or rectangular prismatic ones can be produced by casting or machining or a combination of these processes.

The material can also be shaped in theatre using a scalpel or other tool to enable the implant to be approximated to the desired space or cavity into which it is to be fitted. For applications such as impaction grafting where small fragments are required these can be produced by cutting or breaking pieces of the material to give pieces of the desired size.

For some applications the material can be cut, broken or crushed into small pieces, typically 1 to 10 mm in diameter. Small porous particles of material can be formulated into a coarse paste or putty without loss of their porous architecture. Physiological saline or a solution comprised of one or more biocompatible resorbable polymers can be used to bind small particles of material into pastes or putties. By way of example only, suitable biocompatible resorbable polymers include fibroin, fibrin, collagen, alginate, or synthetic polymers based on monomers of lactic acid, glycolic acid, dioxanone, trimethylene carbonate and caprolactone. Pastes or putties containing material particles may also comprise natural surfactants including by way of example only phospholipids, lysolecithin, or lecithin.

It is to be understood that the material is well suited for applications involving the implantation of porous pieces or porous particles of material whether introduced by impaction grafting or in a paste or putty. This is because the extreme toughness of the particles prevents the moderate stresses produced during implantation from collapsing the open porous structure of the material, maintaining routes for the ingress of mesodermal stem cells or other bone-forming cells into the implanted material.

In a further embodiment, concentrated regenerated fibroin solution is first infiltrated into a fibre lay or between fibres in both cases comprised of resorbable biocompatible fibres before all or part of the regenerated fibroin is gelled and converted to a fibroin-apatite composite. This provides a means of further strengthening and toughening the material. The fibres for this embodiment can be comprised from, by way of example only, silk, collagen, or synthetic polymers based on monomers of lactic acid, glycolic acid, dioxanone, trimethylene carbonate and caprolactone. If silk fibres are used it is advantageous to swell the surface of them first by immersing them for a short period in a chaotropic agent such as lithium bromide and washing away the chaotropic agent before adding the regenerated fibroin. This provides an excellent interface between the fibres and the regenerated fibroin which improves their interaction, strengthening the material when it is gelled.

Devices for anchoring artificial ligaments, tendons or menisci can be formed by forming a twisted or plaited or braided thread, cable or fibre or a plurality of threads, fibres or cables and inserting one end of these into a concentrated fibroin solution. The fibroin solution is then gelled and converted into a fibroin-apatite composite as disclosed above. This ensures that the one or more threads, cables or fibres are firmly anchored into a block of fibroin-apatite composite. A strong anchor can be made by forming a piece of fibroin-apatite composite into a truncated cone with the narrow end of the cone attached to the end or ends of the said thread, cable or cables. To insert the anchor, the cable or cables or fibre or fibres attached to the cone are first passed through a hole drilled through bone or through bone and cartilage. Provided that the hole has a diameter somewhat less than the wide end of the truncated cone a firm anchor point can be made by jamming the cone in the drilled hole. Other geometries including by way of example only a mesa or a wedge can be used to form a firm anchor in this way. A plurality of sub-fibres extending from the main fibre or cable, provide a large surface area to anchor the main fibre or cable into the fibroin-apatite component of the anchor.

By way of example only, such a plurality of fibres for the anchor can be prepared using a modification of the technique used to form pom-poms, such as are used to decorate children's clothing. A small washer-shaped disc typically 5 mm to 10 mm in diameter is cut from a sheet of thin, but stiff material. Multiple turns of a biocompatible and resorbable thread or filament are passed through the central hole of the disc so that they lie radial to the disc. When a sufficient number of turns of thread or filament have been laid down radially, a circumferential cut is made through them at the edge of the disc enabling the disc to be removed and providing an array of radially orientated fibres radiating from a central thread. One to several pom-poms produced in this way can be infiltrated with fibroin and placed in a mould before the fibroin is converted to a fibroin-apatite composite.

### Example 1

### Protocol for the Preparation of Optimised Regenerated Fibroin Solution from Reeled Raw Silk or Silk Cocoons

1. Freshly formed Bombyx mori silk cocoons or reeled raw silk were treated with 10 mM ethylenediaminetetraacetic acid (EDTA) solution for one hour at room temperature (21° C.);

2. The silk cocoons or reeled raw silk was then rinsed in the same solution and thoroughly washed with ultrapure water;

3. The silk cocoons or reeled raw silk was then degummed at 30-40° C. with a trypsin solution at pH 8.5-9.3 in a buffer containing an ammonium salt or ammonia;

4. The silk cocoons or reeled raw silk was thoroughly washed in ultrapure water;

5. The water was squeezed out and the silk cocoons or reeled raw silk was treated with an aqueous 0.1 M to 0.001 M ammonium chloride or ammonium hydroxide solution containing ammonium ions for one hour at 20° C.;

6. The silk cocoons or reeled raw silk was dried overnight at room temperature (21° C.) in conditions of less than 20% humidity and in the presence of anhydrous calcium chloride;

7. The silk cocoons or reeled raw silk was dissolved in an aqueous 9.3M solution of lithium bromide for 4-5 hours with constant stirring at 37° C., at a ratio of 1 g of silk to 5 ml of lithium bromide solution;

8. The resulting fibroin solution was transferred to Visking tubing (molecular weight cut off 12-15 kDa) and dialysed for a minimum of five hours and a maximum of three days against ultrapure water at 5° C. with constant stirring in covered beakers—a large excess of ultrapure water was changed five times at evenly spaced intervals;

9. After dialysis the fibroin concentration in the regenerated silk solution was between 8-10% w/v as determined by gravimetry and/or refractometry—the concentration of the fibroin was increased by leaving the unopened dialysis tubes in a vacuum to obtain a concentration of 8-10% w/v.

### Example 2

### Protocol for the Preparation of Implantable Bone Repair Materials from Optimised Regenerated Fibroin Solution

1. An aqueous 10% w/v Bombyx mori optimized regenerated fibroin solution was prepared as described in Example 1;

2. Aliquots of 20 ml of the solution were dialysed in Visking bags (Molecular Weight Cut-off 12-14 KDa) for two hours at 4° C. against an aqueous buffered solution containing a final concentration of 0.9M dihydrogen sodium phosphate and 1% w/v 2-amino-2-(hydroxymethyl)propane-1,3-diol (Tris) buffer, adjusted to pH 9.0 using 5M sodium hydroxide this step lightly gels the fibroin solution and introduces phosphate ions into the resultant gel;

3. Samples of the gel from step 2) were transferred to a freezer bath for 24 hours at −13° C. to introduce interconnecting pores into the material.

4. While still frozen, samples of the material were cut into pieces with a sharp scalpel and the dialysis bag was removed;

5. The samples of the frozen gel were transferred to an aqueous buffered solution at 37° C. containing a final concentration of 1.5M calcium chloride solution and 1% w/v Tris, adjusted to pH 9.0 with 5M sodium hydroxide to form a fibroin-apatite material;

6. The samples were slowly brought to 50% ethanol in one day to remove excess salts and convert the protein into the Silk II state;

7. The samples were brought to dry ethanol over two days;

8. The samples were vacuum dried at 40° C. and transferred to pure dry hexamethylene di-isocyanate at 80° C. under dry nitrogen for two days;

9. Excess hexamethylene di-isocyanate was removed from the material as follows:
a) Four rinses with cold anhydrous acetone followed by refluxing in anhydrous acetone overnight at 60° C.;
b) Water added to hydrolyse any remaining CNO groups;
c) Material dried in an oven initially at 40° C. and finally at 60° C. before autoclaving (no trace of acetone could be detected by smell);
d) checked for the absence of the CNO peak in the material using fourier transform infra red (FT-IR) spectroscopy.

### Example 3

### Protocol for Testing Fibroin-Apatite Materials

### Mineralisation

Mineral loadings were determined gravimetrically by heating of the material to 500° C. in air. The preferred embodiment gave loadings of 30% w/w mineral content while modification of the protocol, as described above, gave mineral loadings up to 70% w/w mineral content.

Samples of the material were further studied by scanning electron microscopy (JEOL JSM 6330) fitted with an energy dispersive X-ray analyser. X-ray energy spectra demonstrated the co-localization of calcium and phosphate within the pore walls (FIGS. 5 and 6) and the presence of high levels of mineralization with calcium phosphate (FIG. 7). Evidence of small quantities of chloride ions in the X-ray energy spectra may be accounted for by chlor-substitution of the hydroxyapatite (see below).

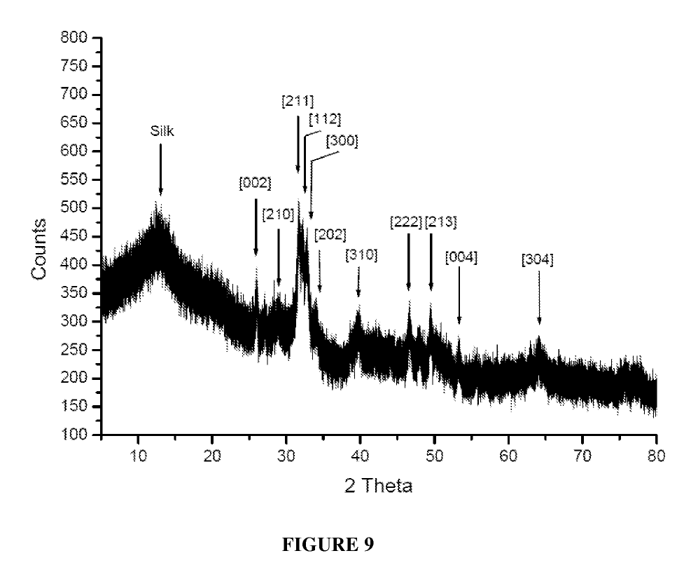

FT-IR spectroscopy (KBr discs; Perkin-Elmer Spectrum 1) confirmed the presence of large quantities of phosphate in the composite (FIG. 8 peaks E and F). Powder X-ray diffraction (Bruker D8) demonstrated the presence of chloride-substituted hydroxyapatite in the composite (FIG. 9).

### Example 4

### Protocol for Testing Fibroin-Apatite Materials

### Load-Bearing Properties

Mechanical tests (Zwick 1478) were performed on fully hydrated samples of the material, which were cut into cylinders and compressed with a crosshead speed of 2 mm min⁻¹ to destruction.

The stress/strain curve (FIG. 10) shows that the material has an extended plastic deformation phase.

The mean unconfined compressive toughness of the di-isocyanate cross-linked material was 11.93±8.40 kJ m⁻², n=6 (obtained using the J-integral method).

The mean unconfined ultimate compressive strength (stress to yield point) of the material was 14 MPa (n=5).

The unconfined compressive elastic modulus of the material was 175 MPa (n=5).

In the case of compressive strength and the compressive elastic modulus, the measured values are reasonably close to the target values for a BRM, being 20 MPa for the compressive strength and 100-500 MPa for the compressive elastic modulus, respectively.

In the case of toughness, the measured value exceeded target values understood to be advantageous for a BRM, the target value being 1.3 kJ m⁻³.

### Example 5

### Protocol for Testing Fibroin-Apatite Materials

### Pyrogenicity

5 mg samples of the material were inserted into pyrogen-free 1.5 ml polypropylene reaction vials (Eppendorf) with heat-sterilized forceps together with 1000 μl of isotonic saline solution (Berlin-Chemie AG) and either 100 μl of LPS spike (NIBSC, UK; WHO reference, Escherichia coli, 0113:H10) diluted in saline, or 100 μl of saline as a control.

Spiking the samples with LPS (1 or 4 EU/ml) was used to exclude interference from blood monocyte activities, for example from toxic or immuno-modulatory samples. Spike recovery values of between 50-200% were deemed acceptable to exclude interference.

A standard curve for endotoxin diluted in saline with 0.5 EU/ml as the threshold concentration for pyrogenicity was included in all tests.

100 μl of pooled blood obtained from healthy volunteers and checked for infections by differential blood cell counting (Pentra 60, ABX Diagnostics, France) was added to each reaction vial to give a final incubation volume of 1200 μl and left for 21-24 hours at 37° C. and 5% CO².

Cell-free supernatants were obtained by centrifugation at 13,000 rpm for two minutes and assayed immediately, or stored at −80° C. until measurements could be taken.

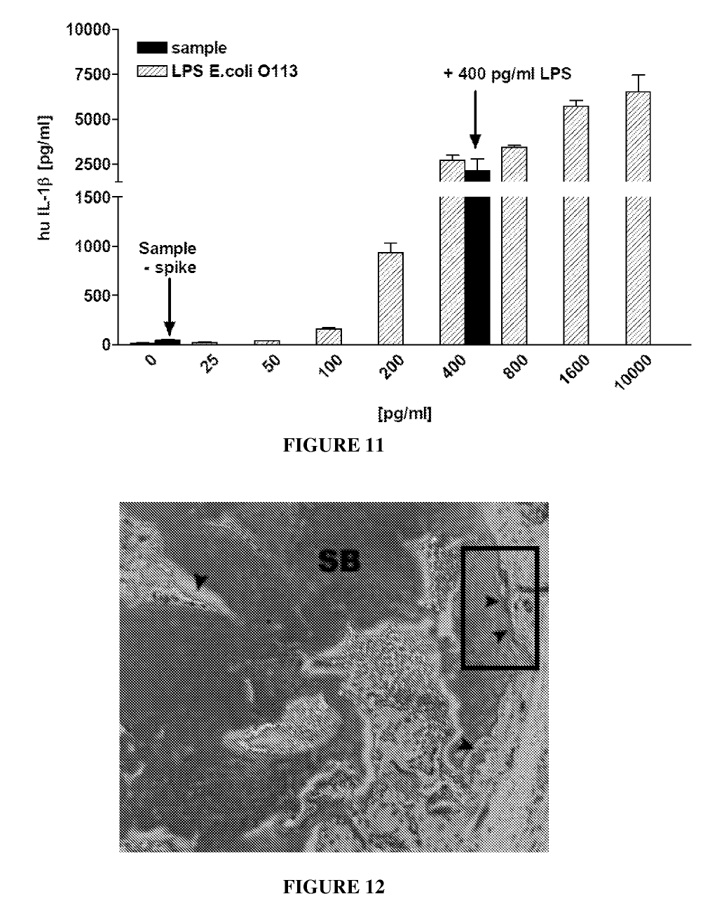

Release of IL-1 was detected by ELISA with an antibody pair and recombinant standard (R&D Systems, Wiesbaden, Germany) The detection limit of the ELISA was 3.5 pg/ml IL-1β. The assay demonstrated that the pyrogenicity of the material was negligible (FIG. 11).

### Example 6

### Protocol for Testing Fibroin-Apatite Materials

### Osteogenicity

Adult human bone marrow samples were obtained from haematologically normal patients undergoing routine hip replacement surgery for osteoarthritis. Only tissue that would have been discarded was used with the approval of the Southampton and South West Hampshire Local Research Ethics Committee. A total of four samples (two male and two female of mean age 70±13 years) were prepared.

Primary cultures of bone marrow cells were established, after enrichment by selection for STRO-1 (a marker, from a CD34+ fraction, of pluripotency) using STRO-1 antibody hybridoma supernatant (gift from Dr J Beresford, University of Bath, UK), which facilitates rapid expansion in vitro prior to implantation (S. Gronthos, S. E. Graves, S. Ohta, P. J. Simmons, Blood 84, 4164-4173 (1994)).

Cultures were maintained in basal medium (MEM with 10% FCS, 1% penicillin/streptomycin) at 37° C. in humidified air with 5% CO².

At 70% confluence, osteogenic media (basal media plus 10 nmol/L dexamethasone plus 100 nmol/L ascorbate-2-phosphate) was substituted and after a further 24 hours, cells were gently trypsinised, counted and resuspended in osteogenic media in preparation for seeding onto material samples.

Tissue culture reagents were obtained from Gibco/BRL (Paisley, Scotland). Reagents were of analytical grade from Sigma Chemical (Poole, UK) unless otherwise stated.

3 mm cubes of fully hydrated autoclaved material were soaked in basal media for 24 hours and transferred to 24-well tissue culture plates.

48 hours prior to implantation, 10 μl of a cell suspension [1×104 cells] of adult human bone marrow stromal cells was pipetted onto each cube and incubated at 37° C. for 30 minutes before 1 ml of osteogenic media was added to each well.

Unseeded material samples were used as controls.

After specified intervals, material was fixed in buffered formaldehyde solution and embedded in methacrylate resin. 10 μm sections were cut with a tungsten knife.

Fluorescent staining with cell tracker green and ethidium homodimer-1, as well as histological staining with haematoxylin and eosin, indicated the presence of viable HBMSCs (human bone marrow stromal cells) within the pores and on the surface of the fibroin-apatite material. Inward growth of the cells was visible by day three and complete colonisation of the porous monolith observed after seven days.

The HBMSCs remained viable over three weeks in culture, with maintenance of the osteoblast phenotype within the material, as evidenced by type I collagen and alkaline phosphatase immunocytochemistry.

### Example 7

### Protocol for Testing Fibroin-Apatite Materials

### In Vivo Testing

3 mm cubes of fully hydrated autoclaved material were seeded with human bone marrow stromal cells. They were implanted without prior incubation subcutaneously into eight immunocompromised MFI nu/nu mice under anaesthesia.

Seeded samples were placed in the left flank and unseeded controls in the right flank of each animal Mice were left for 4, 8 and 12 weeks before sacrifice.

Haematoxylin and eosin stained glycol methacrylate resin sections of cell-seeded material taken from the mice were examined (FIGS. 12-15). After eight weeks, the sections showed the presence of newly formed bone secreted by osteoblasts on the surface of the porous material. Palisades of osteoblasts were observed on the osteoid surface and connective tissue scale. Evidence of remodeling of the newly formed bone by multinucleate osteoclasts was observed on the surface of the osteoid matrix. No evidence of adverse cell or tissue reactions was observed in seeded and unseeded controls.

These observations together with those of in vitro testing described above, demonstrate the excellent biocompatibility of the di-isocyanate cross-linked material. The observations made on in vivo testing further strongly suggests that the material is highly osteogenic, that the material is slowly resorbed and that the bone formed de novo in the material undergoes remodeling.

### Observations

The porous, resorbable, biocompatible, pyrogen-free, implantable material described above is highly advantageous, because it combines the properties of compressive strength, compressive elastic modulus and compressive toughness close to that of previously defined target valves with an appropriate resorption rate and excellent tissue regenerative properties. These properties make the material suitable for all immediate and non-immediate load-bearing applications, non-load-bearing applications and as a substitute for allograft and autograft bone.

The similarity of the mechanical properties of the implantable material to those of natural bone make the material capable of immediately bearing the stresses to which bones are subjected in normal movement, thereby avoiding the need for prolonged periods of bed rest and minimizing the use of internal or external supports. The implantable material can therefore, be used in load-bearing implant locations to replace all or a part of a bone, or to lie between a bone and a metallic or ceramic or plastic prosthesis.

The exceptional toughness of the implantable material makes it particularly suited to impaction grafting, because the pores are protected from collapse during impaction allowing for rapid ingress of cells and blood vessels. Therefore, the implantable material can also be used to fill voids in bones.

The high and open porosity and large mean pore size of the implantable material enables mesenchymal stem cells, osteoblasts, osteoclasts and developing capillaries to migrate into the material initiating the materials conversion to natural bone. This together with the excellent biocompatibility and adhesiveness for cells of the implantable material allows cells to adhere, grow and differentiate within the pores of the material enabling the rapid de novo production of bone.

The slow resorbability of the implantable material enables it to be gradually and completely replaced by functional endogenous bone.

1. A method for the preparation of an implantable material for the repair, augmentation or replacement of bone from a fibroin solution, the method comprising the steps of:
preparing a gel from the fibroin solution; and
preparing a material by subjecting the gel to one or more steps of freezing and thawing the gel,
wherein the step of preparing the gel from the fibroin solution is performed in the presence of phosphate ions.

2. The method according to claim 1, wherein the method comprises the further step of subsequently treating the material with a cross-linking agent.

3. A method for the preparation of an implantable material for the repair, augmentation or replacement of bone from a fibroin solution, the method comprising the steps of:
preparing a gel from the fibroin solution;
preparing a material by subjecting the gel to one or more steps of freezing and thawing the gel,
wherein the method comprises the further step of subsequently treating the material with an isocyanate.

4. The method according to claim 3, wherein the gel is treated with phosphate ions, or the step of preparing the gel from the fibroin solution is performed in the presence of phosphate ions.

5. (canceled)

6. The method according to claim 1, wherein the step of preparing the gel from the fibroin solution comprises a gelling reagent containing phosphate ions.

7. The method according to claim 2, wherein the material is treated with calcium ions to form a fibroin-apatite before treating the material with the cross-linking agent.

8. (canceled)

9. The method according to claim 7, wherein the calcium ions are provided by a solution of calcium chloride.

10. The method according to claim 9, wherein the material is washed with ethanol to remove excess calcium chloride and to convert the fibroin into a silk II state.

11. The method according to claim 10, wherein the material is dried after the washing step.

12. The method according to claim 2, wherein the cross-linking agent includes one or more of hexyl isocyanate (HMI), methyl isocyanate (MIC), hexamethylene di-isocyanate (HDI), methylene diphenyl di-isocyanate (MDI), toluene di-isocyanate (TDI) and isophorone di-isocyanate (IPDI).

13. The method according to claim 2, wherein the treatment with the cross-linking agent is carried out with substantially no fibroin swelling agents.

14. (canceled)

15. The method according to claim 2, wherein the method comprises the further step of removing excess cross-linking agent from the material in one or more rinsing steps.

16. (canceled)

17. The method according to claim 15, wherein the method comprises the further step of drying the material.

18. (canceled)

19. The method according to claim 1, wherein the fibroin solution is a regenerated fibroin solution.

20. The method according to claim 19, wherein the regenerated fibroin solution is prepared by a method comprising treating silk or silk cocoons with an ionic reagent comprising an aqueous solution of monovalent cations and monovalent anions, the cations and anions having ionic radii of at least 1.05 Angstroms and a Jones-Dole B coefficient of between −0.001 and −0.05 at 25° C.

21. A method for the preparation of an implantable material for the repair, augmentation or replacement of bone from a regenerated fibroin solution, wherein the regenerated fibroin solution is prepared by a method comprising step of treating silk or silk cocoons with an ionic reagent comprising an aqueous solution of monovalent cations and monovalent anions, the cations and anions having ionic radii of at least 1.05 Angstroms and a Jones-Dole B coefficient of between −0.001 and −0.05 at 25° C.

22-31. (canceled)

32. An implantable repair, bone augmentation, or bone replacement material obtainable by the method according to claim 1.

33-47. (canceled)
